(19) United States
(12) Patent Application Publication
(10) Pub. No.: US 2012/0219629 A1
(43) Pub. Date: Aug. 30, 2012

(54) COMPOSITIONS AND METHODS FOR CONTROLLED DELIVERY OF COMPOUNDS

(75) Inventor: Seth Miller, Englewood, CO (US)

(73) Assignee: EMPIRE TECHNOLOGY DEVELOPMENT LLC

(21) Appl. No.: 13/382,511

(22) PCT Filed: Feb. 25, 2011
(86) PCT No.: PCT/US2011/026348

§ 371 Date: Jan. 5, 2012

(51) Int. Cl. A61K 38/18; A61K 9/50; A61K 9/51; C12N 5/02; A61K 47/32; A61P 15/00; A61P 9/00; A61K 38/19; A61K 47/34

(52) U.S. Cl. 424/486; 514/8.1; 514/7.6; 514/772.3; 514/772.4; 435/375

(57) ABSTRACT

Disclosed are methods, compositions and kits pertaining to controlled delivery of compounds. In certain aspects and embodiments the present technology relates to compositions and methods for controlled delivery of a compound such as a bioactive compound which involve exposing a matrix comprising the bioactive compound, a crosslinkable monomer and a polymerization initiator to an external stimulus; wherein the external stimulus causes crosslinking of the matrix. In some embodiments, the crosslinking causes a decrease in the release of the compound from the matrix.

### TECHNICAL FIELD

This disclosure relates generally to methods, kits and compositions pertaining to controlled delivery of compounds.

### BACKGROUND

The following description is provided to assist the understanding of the reader. None of the information provided or references cited is admitted to be prior art.

The ability to control release of compounds such as bioactive compounds is desirable in a number of various types of settings. Kempen et al., Journal of Biomedical Materials Research Part A (2004), 70A:293-302 (hereby incorporated by reference in its entirety) disclose biodegradable controlled release microspheres made from a blend of poly(propylene fumarate) and poly(lactic-co-glycolic acid). U.S. Pat. No. 6,884,432 (hereby incorporated by reference in its entirety) discloses microspheres for controlled release of a bioactive agent, including microspheres based on poly(propylene fumarate) for immobilization and controlled drug delivery.

### SUMMARY

The present technology disclosed herein is based at least in part on the discovery of methods and compositions that may be used to control release of compounds, including bioactive compounds.

In one aspect, provided are methods and compositions for controlled delivery of a compound (for example, a bioactive compound). In some embodiments, provided are methods that may include exposing a matrix that includes the compound, a crosslinkable monomer and a polymerization initiator to an external stimulus wherein the external stimulus causes crosslinking of the matrix, as well as compositions useful in such methods.

In other aspects provided are compositions that include: a matrix configured to release the bioactive compound, one or more crosslinkable monomers and a polymerization initiator configured to initiate polymerization of the crosslinkable monomer in response to an external stimulus. In certain embodiments, the polymerization initiator is or includes a photoinitiator. In certain embodiments the matrix may be included within, or in the form of, microcapsules or nanocapsules.

In certain embodiments, the compound of the present technology is one or more of a biologically active compound, a cytokine, a growth factor, or VEGF.

In some embodiments, the crosslinking causes a decrease in the rate that the bioactive compound is released from the matrix. For example, in certain illustrative embodiments the compound is released from the matrix prior to crosslinking and the crosslinking causes a reduction in the rate that the bioactive compound is released from the matrix. In some embodiments the rate of release of the compound from the matrix following crosslinking is less than 50% of the rate of release of the bioactive compound before crosslinking; or less than less than 25% of the rate of release of the bioactive compound before crosslinking; or less than 10% of the rate of release of the bioactive compound before crosslinking; or less than 5% of the rate of release of the bioactive compound before crosslinking.

In some embodiments of the present technology, the matrix is administered to a subject or a cell prior to crosslinking. In certain embodiments the subject is a mammal; in some embodiments the subject may be a human. In various embodiments the matrix may be administered to a cell; for example a cell cultured in vitro. In some embodiments, the matrix is administered to a cell in vivo, for example the matrix may be administered to a cell present in a subject in situ. In some embodiments the matrix is administered to a subject or a cell prior to crosslinking and the matrix is then exposed to the external stimulus at least 1 hour after administration; or at least 6 hours after administration; or at least 12 hours after administration; or at least 24 hours after administration.

In various illustrative embodiments of the methods and compositions of the present technology, the crosslinkable monomer of the matrix may be a biodegradable crosslinkable monomer. In some embodiments, the crosslinkable monomer includes one or more of: propylene fumarate, DL-lactic-co-glycolic acid or diacrylated poly(ethylene glycol) (PEGDA) monomers. In some embodiments, the crosslinkable monomer includes both propylene fumarate and DL-lactic-co-glycolic acid.

In some embodiments of the present technology, the polymerization initiator of the matrix is a photoinitiator, for example a two-photon photoinitiator. In some embodiments the wherein the polymerization initiator is one or more selected from the group consisting of 2-hydroxy-1-[4-(2-hydroxyethoxy) phenyl]-2-methyl-1-propanone (I2959); 9,10-bis-pentyloxy-2,7-bis[2-(4-dimethylamino-phenyl)-vinyl]anthracene (BPDPA) or a mixture of riboflavin and L-arginine.

The external stimulus of the compositions and methods of the present technology may in some embodiments be light. In certain embodiments, the external stimulus is ultraviolet light; in some embodiments the external stimulus is light at a wavelength greater than 700 nm. The light may be applied using a rasterizing laser and/or a photomask. The light as an external stimulus may be applied to a localized area of a subject. For example, a method according to the present technology may include administering a matrix as described herein to a subject and administering light as an external stimulus to a localized area of the subject. In some embodiments, the localized light is applied to an area of the subject that is different than the area of administration.

In one aspect, provided are kits that includes a composition or matrix in accordance with the present technology. In some embodiments the kit further includes instructions for use, for example the instructions may include instructions to administer the composition to a cell or subject, and stimulate the composition with light.

In another aspect, included within the present technology is a method of manufacturing compositions for controlled delivery of a compound such as described herein. The method may include forming microcapsules or nanocapsules that include a compound for controlled delivery, a crosslinkable monomer and a polymerization initiator (such as a photoinitiator). In some embodiments the crosslinkable monomer is a multifunctional monomer incorporating acrylates, methacrylates, acrylimides, styryls, or the like. In some embodiments, the crosslinkable monomer includes one or more of: propylene fumarate; DL-lactic-co-glycolic acid; or DL-lactic-co-glycolic acid or diacrylated poly(ethylene glycol) (PEGDA) monomers. The polymerization initiator may be one or more of: 2-hydroxy-1-[4-(2-hydroxyethoxy) phenyl]-2-methyl-1-propanone (I2959); 9,10-bis-pentyloxy-2,7-bis[2-(4-dimethylamino-phenyl)-vinyl]anthracene (BPDPA) or a mixture of riboflavin and L-arginine.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 is from TP Richardson et al., Nature Biotechnology (2001) 19:1029-1034 and shows that when VEGF encapsulated in PLA without crosslinking by a polymerization initiator and external stimulus of the present technology, the VEGF is released quickly in the first week, followed by a slow steady release over subsequent weeks.

FIG. 2 is from M. A. Vandelli, et al., International Journal of Pharmaceutics (2001) 215:175-184 and illustrates a plot of drug release over time for gelatin microsphere with different crosslinking levels. As crosslinking of the microsphere increases, the release amount and release rate decrease. Starred line represents the least crosslinked microsphere; upside-down triangles the most.

FIG. 3 illustrates the polymerization of PPF occurring through crosslinking of its internal double bonds.

FIG. 4 is from Jin-Feng Xing et al., App. Phys. Lett (2007) 90:131106 and shows photoinitiator BPDPA that absorbs at 800 nm in a two-photon process.

### DETAILED DESCRIPTION

In the following detailed description, reference may be made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Unless otherwise stated, the singular forms “a ” “an,” and “the” as used herein include plural reference.

### Controlled Delivery of Compounds

Controlled delivery of compounds, such as biologically active compounds, may be desirable for many reasons. For example, living tissue releases an array of cytokines to initiate cell differentiation, growth, maturation, repair, and other functionality. In vivo, cytokine release is governed by a complex homeostasis that regulates this chemical crosstalk, assuring that a precise dose of chemical is released at the correct place and time. For in vitro tissue growth applications, cytokines may be delivered artificially, for instance by controlled-release matrix.

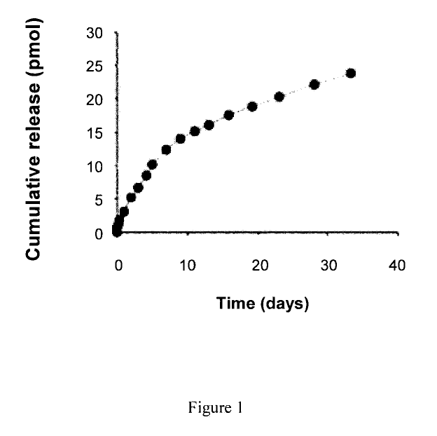

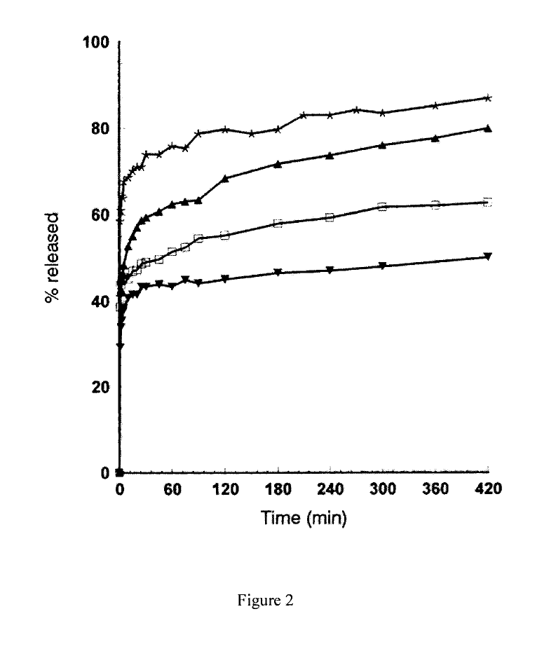

Many current methods for controlling drug release involve encasing the compound in a polymeric matrix material designed to decompose at a desired rate. Using such current methods it is often not possible to control the initial release rates, steady-state rates, and final rates of delivery to mimic what is accomplished by homeostasis. In this regard, the release kinetics of encapsulated compounds can be controlled somewhat by varying the nature of the biodegradable encapsulant. For example, a more hydrophobic encapsulating agent (A) degrades more slowly than a more hydrophilic one (B) (all other things being equal), and thus drug is released more slowly from A than B. Similarly, a polymer of higher molecular weight will decompose into soluble fractions more slowly than a polymer of low molecular weight, resulting in slower release. These chemical properties can thus be tuned in order to control the release of a drug to tissue. Generally the release of the drugs follows a typical exponential decay pattern, but with careful polymer design the release can be made linear over relevant time periods. However, after this linear portion is complete, the polymer still holds a considerable amount of drug, which is continually released over a longer time period. This is demonstrated in FIG. 1, which shows the release profile for the growth factor VEGF encapsulated in a PLA matrix. Crosslinking a matrix material can have a significant effect on degradation kinetics. For example, FIG. 2 shows a decrease in drug release rate as crosslinking increases in gelatin microspheres. Increased crosslink density results in a significant drop in the slope of the plot, indicating a dramatic decrease in the availability of the drug.

Halting delivery of bioactive compounds, such as a cytokine, may be desired for a number of reasons. For example, in the early stages of vascularization, vascular endothelial growth factor (VEGF) is needed to assist in growth of a vascular network, while platelet-derived growth factor (PDGF) assists in its maturation. Thus, after growth is essentially completed, it may in many circumstances be desirable to turn off the delivery of VEGF as is done in vivo, rather than wait for such delivery to tail off slowly as is current practice. Further, it may be that growth of a particular tissue occurs non-homogenously across the sample, for instance to due to irregularities in mass transport of nutrients or other signaling molecules.

Accordingly, in many embodiments of the present technology, compositions and methods are provided that allow for one to turn off or suddenly decrease delivery of a compound when it is no longer desired, mimicking the behavior of native cells. In many embodiments, the delivery is stopped or decreased using an external stimulus, for example, the external stimulus may be exposure to light. In some illustrative embodiments the stimulus may be chemical, heat, a magnetic field or an electric field. As such, the present technology in various embodiments provides methods and compositions to controllably halt the diffusion of compounds from a matrix when they are no longer desired. In certain illustrative examples, a matrix comprising a biodegradable crosslinkable monomer such as propylene fumarate, or any other applicable monomer as known in the art, may be loaded with a compound such as a cytokine and a polymerization initiator, such as a photoinitiator. This slow release formulation is administered to a cell, tissue or subject for delivery of the compound. When it is desired to halt the delivery of the cytokine, matrix crosslinking may be initiated by an external stimulus such as UV light. In such examples crosslinking dramatically slows the degradation rate of the matrix, and may also slow diffusion of the bioactive compound through the matrix, enabling delivery to be brought effectively to a halt. Release of the compound can thus be limited by an external stimulus, and can therefore be modulated as desired.

Accordingly, in various aspects and embodiments of the present technology, methods and compositions are provided in which a matrix containing a compound, a polymerizable or crosslinkable monomer and optionally a photoinitiator are manufactured. The matrix is optionally formed into nanocapsules and/or microcapsules. The matrix may be administered to a subject or cell and allowed to release the compound (either by conventional diffusion or by use of a triggering stimulus). Once it is desired to reduce or halt release of the compound the matrix may be exposed to an external stimulus (such as, but not limited to, light) that crosslinks or polymerizes the polymerizable or crosslinkable monomer and, in turn, reduces release of the compound from the matrix. In certain embodiments, following crosslinking, the matrix will remain biodegradable, but its rate of degradation will decrease to the point that the compound release drops below a therapeutic threshold. In some embodiments the rate of release of the compound from the matrix following crosslinking is less than 50% of the rate of release of the bioactive compound before crosslinking; or less than 25% of the rate of release of the bioactive compound before crosslinking; or less than 10% of the rate of release of the bioactive compound before crosslinking; or less than 5% of the rate of release of the bioactive compound before crosslinking. In some embodiments, the external stimulus is applied (and hence, crosslinking is initiated) at least 1 hour after administration of the matrix to the cell or subject; or at least 2 hours after administration; or at least 6 hours after administration of the matrix to the cell or subject; or at least 12 hours after administration of the matrix to the cell or subject; or at least 24 hours after administration of the matrix to the cell or subject; or at least 2 days after administration of the matrix to the cell or subject; or at least 3 days after administration of the matrix to the cell or subject; or at least 4 days after administration of the matrix to the cell or subject; or at least 5 days after administration of the matrix to the cell or subject; or at least 6 days after administration of the matrix to the cell or subject; or at least 1 week after administration of the matrix to the cell or subject; or at least 2 weeks after administration of the matrix to the cell or subject. In some embodiments, the external stimulus is applied (and hence, crosslinking is initiated) approximately 1 hour after administration of the matrix to the cell or subject; or approximately 2 hours after administration; or approximately 6 hours after administration of the matrix to the cell or subject; or approximately 12 hours after administration of the matrix to the cell or subject; or approximately 24 hours after administration of the matrix to the cell or subject; or approximately 2 days after administration of the matrix to the cell or subject; or approximately 3 days after administration of the matrix to the cell or subject; or approximately 4 days after administration of the matrix to the cell or subject; or approximately 5 days after administration of the matrix to the cell or subject; or approximately 6 days after administration of the matrix to the cell or subject; or approximately 1 week after administration of the matrix to the cell or subject; or approximately 2 weeks after administration of the matrix to the cell or subject. In some embodiments, the external stimulus is applied (and hence, crosslinking is initiated) between 30 minutes and 2 hours after administration of the matrix to the cell or subject; or between 1 hour and 3 hours after administration; or between 4 hours and 10 hours after administration of the matrix to the cell or subject; or between 10 hours and 18 hours after administration of the matrix to the cell or subject; or between 18 hours and 36 hours after administration of the matrix to the cell or subject; or between 1 day and 3 days after administration of the matrix to the cell or subject; or between 3 days and 4 days after administration of the matrix to the cell or subject; or between 4 days and 5 days after administration of the matrix to the cell or subject; or between 5 days and 6 days after administration of the matrix to the cell or subject; or between 6 days and 7 days after administration of the matrix to the cell or subject; or between 1 week and 2 weeks after administration of the matrix to the cell or subject; or between 2 weeks and 3 weeks after administration of the matrix to the cell or subject.

Using the methods and compositions of the present technology, the delivery of compounds such as biologically active compounds may be turned off selectively in one region, while allowing unaffected regions to continue to receive these chemical signals. For example, in such embodiments, an external stimulus such as light may be applied locally to a subject or cell such as through the use of a rasterizing laser or photomask so that delivery of the compound is halted in one region but allowed to continue in one or more other regions. For example in one example of an illustrative embodiment, compositions of the of the present technology (e.g., a matrix, a nanocapsule or a microcapsule) may be embedded within or bound to a tissue engineering scaffold and the external stimulus (such as light) may be applied to only a portion of the scaffold or growing tissue, thus halting release of the compound only in the area exposed to the stimulus. In another illustrative embodiment, compositions of the of the present technology (e.g., a matrix, a nanocapsule or a microcapsule) may be administered to a subject systemically and the external stimulus (such as light) may be selectively applied to certain areas, regions or organs of the subjects body to halt or reduce release of the compound only in such localized areas. In some embodiments, in which the composition and stimulus is administered to a subject or cell, the stimulus may be applied in a manner that minimizes harm to the cells or tissues; or to cells or tissues different than those targeted by the stimulus. In embodiments where the stimulus is light this may be done, for example, by modulating the wavelength and/or intensity of the light, and/or using a focused light (e.g., a laser) or photomask such as to achieve sufficient stimulation to crosslink the composition in the desired area while minimizing harm to cells or tissues. In some embodiments, the light may be at a wavelength that is greater than 400 nm, or greater than 500 nm, or greater than 600 nm, or greater than 700 nm. In some illustrative embodiments the wavelength is between 700 and 1000 nm, or between 700 and 800 nm. In some embodiments in which the light needs to travel through cells or tissue, a wavelength between 700 and 1000 nm, or between 700 and 800 nm may be selected because light at this wavelength may be able to penetrate certain tissue without scattering and/or because light at higher wavelengths may be less likely to cause DNA damage. In some embodiments, the light source is a femtosecond pulsed laser suitable for use in two-photon applications; and the light wavelength is between 700 and 1000 nm, or between 700 and 800 nm.

In some embodiments the external stimulus is a magnetic field, electrical field or heat. In certain illustrative embodiments where the external stimulus is a magnetic field, electrical field or heat, the a polymerization initiator or crosslinking agent may be contained within a hydrogel that is in or surrounding the matrix. The hydrogel has a melting point slightly above the body temperature or culture temperature where the matrix is administered. The electric field or heat applied as an external stimulus causes the hydrogel to melt and release the polymerization initiator or crosslinking agent, which in turn causes the matrix to crosslink and reduce or halt release of the bioactive compound. In certain embodiments, hydrogel includes superparamagnetic nanoparticles (see, for example, J Dobson, Gene Therapy(2006)13:283-287) disposed throughout the hydrogel, the external stimulus is a magnetic field, and the application of the magnetic field external stimulus causes the superparamagnetic nanoparticles to heat, thus melting the hydrogel, releasing the polymerization initiator or crosslinking agent and crosslinking the matrix. In some illustrative such embodiments the crosslinking agent is DSP (Dithiobis[succinimidyl propionate]) and the crosslinkable monomer contains one or more free amine units. In certain illustrative embodiments, the magnetic field, electrical field or heat is applied such that the temperature of the hydrogel reaches a temperature that is at least 4° C.; or at least 5° C.; or at least 6° C.; or at least 7° C.; or at least 8° C.; or at least 9° C.; or at least 10° C. higher than the culture temperature or body temperature where the matrix is administered. For example, if the culture temperature or body temperature is 37° then the magnetic field, electrical field or heat is applied such that the temperature of the hydrogel reaches at least 41° C.; or at least 42° C.; or at least 43° C.; or at least 44° C.; or at least 44° C.; or at least 45° C.; or at least 46° C.; or at least 47 ° C. In certain illustrative embodiments, the external stimulus may be applied as described in Derfus, et. al., Advanced Materials, 19:3932-3936 (2007), hereby incorporated by reference in its entirety. For example, in certain illustrative embodiments the external stimulus may be an electromagnetic field applied with a 3 kW power supply for a period of about 5 minutes.

In some embodiments the external stimulus is a chemical. In certain illustrative embodiments where the external stimulus is a chemical, the chemical is added to the subject or culture and causes the matrix to crosslink. In some illustrative embodiments the chemical external stimulus is glutaminase and the crosslinkable monomers include amines and or glutamine. In other illustrative embodiments the matrix includes cystine amino acids or other thiols and the chemical external stimulus is a chemical oxidant, such as but not limited to hydrogen peroxide (for example dilute hydrogen peroxide) or an enzymatic oxidant such as a protein disulfide isomerase.

### Crosslinkable Monomers and Polymerization Initiators.

The compositions of the present technology (e.g., a matrix, a nanocapsule or a microcapsule) may include a crosslinkable monomer. A “crosslinkable monomer” as used herein is any monomer or chemical that can polymerize or crosslink, for example in the presence of an external stimulus. In certain embodiments, the crosslinkable monomer may interact with or respond to a polymerization initiator if present in the composition.

The concept may be illustrated by an embodiment that uses photocrosslinking of a polymer matrix to halt delivery of compounds (for example biologically active molecules such as cytokines) trapped within that matrix. In one preferred embodiment, this is performed using a polymer containing propylene fumarate monomers. This polymer can be pure poly(propylene fumarate) (PPF), or copolymers containing another monomer such as lactic acid along with propylene fumarate.

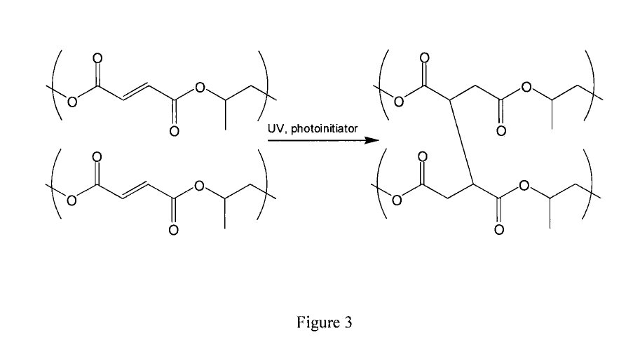

Crosslinkable PPF systems have been developed into microcapsules for drug delivery, for instance as applied in U.S. Pat. No. 6,884,432, hereby incorporated by reference in its entirety. In various illustrative embodiments of the present technology PPF monomers may be formulated into a controlled release matrix as disclosed herein. For example, FIG. 3 illustrates how PPF can be polymerized by a radical initiator (UV light and/or a photoinitiator) that leads to crosslinking via its internal double bonds. Accordingly, PPF monomers and a photoinitiator may be included with a compound (for example a biological compound) in a matrix, microparticle or nanoparticle of the present technology, and once the matrix microparticle or nanoparticle is exposed to UV light the PPF monomers will polymerize resulting in a decrease in the release of the compound from the matrix, microparticle or nanoparticle. In such embodiments, upon crosslinking, the biodegradation of the PPF polymer will slow considerably, and the tortuosity for diffusion of the controlled release compound will increase substantially. Both of these effects will serve to significantly drop the rate of delivery of the compound from the PPF matrix, turning off the biological efficacy of the agent.

Other crosslinkable or polymerizable monomers may also be used in conjunction with the present technology. In some embodiments, the crosslinkable monomer includes one or more of: propylene fumarate, DL-lactic-co-glycolic acid or diacrylated poly(ethylene glycol) (PEGDA) monomers. In some embodiments, the crosslinkable monomer includes both a polymerizable moiety including a monomer or monomers such as but not limited to propylene fumarate and a non-polymerizable moiety such as but not limited to DL-lactic-co-glycolic acid.

In some circumstances, a PPF co-polymer with other materials (such as polylactic acid) is used, as this may allow tuning of the biodegradation rate of the matrix. In such embodiments the capsule may be designed similar to a traditional controlled-release formulation, so that the drug will be released at a desired rate before the ‘off’ switch it triggered through the present technology. As long as there is some PPF monomer in the copolymer, the compositions may be used as in the present technology, without substantively impacting any other desired parameters of the drug delivery system.

The polymerization and/or crosslinking process may in certain embodiments benefit from the use of a radical photoinitiator to transduce light into the chemical crosslinks. In some embodiments, the photoinitiator will be effective with light with a wavelength >700 nm, or >725 nm, or >750 nm, or >775 nm, or about 800 nm. In certain embodiments the wavelength used penetrates the tissue of the cells or subject without scattering (i.e., longer wavelengths may penetrate further without scattering. In some embodiments the photoinitiator is a two-photon photoinitiator, as two-photon processes may under certain conditions allow for improved spatial resolution, so that inactivation can be accomplished in only a small part of the construct if desired.

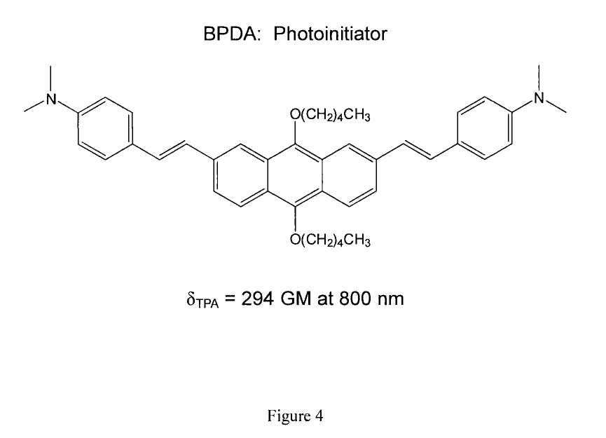

The chemical 9,10-bis-pentyloxy-2,7-bis[2-(4-dimethylamino-phenyl)-vinyl]anthracene (BPDPA), an illustrative embodiment of a two-photon process photoinitiator, is shown in FIG. 4. Any of many other photoinitiators may also be used. In certain embodiments, a photoinitiator may be selected based on the wavelength it absorbs light. For example 2-hydroxy-1-[4-(2-hydroxyethoxy)phenyl]-2-methyl-1-propanone (I2959) such as described in Fairbanks, et al., Biomaterials (2009) 30:6702-6707 (hereby incorporated by reference in its entirety) is another photoinitiator that may be used. In other embodiments, riboflavin may be used as a photoinitiator, with or without the inclusion of L-arginine as a co initiator or with a mixture of riboflavin and L-arginine as a co-initiator (see for example, Kim et al., Journal of Biomedical Materials Research Part B: Applied Biomaterials (2009), 91B:390-400, hereby incorporated by reference in its entirety).

### Nanocapsules and Microcapsules

In some embodiments, the matrix material of the delivery system and methods of the present technology is in the form of microcapsules or nanocapsules.

The term “nanocapsule,” “nanoparticle” or “nanosphere” as used herein refers to particles having a size (e.g., a diameter) between 1 nm and 1,000 nm; or between 1 nm and 600 nm; or between 50 nm and 500 nm; or between 100 nm and 400 nm; or between 150 nm and 350 nm; or between 200 nm and 300 nm. In certain embodiments, a “nanocapsule composition” as used herein refers to a composition that includes particles wherein at least 30%; or at least 40%; or at least 50%; or at least 60%; or at least 65%; or at least 70%; or at least 75%; or at least 80%; or at least 85%; or at least 87%; or at least 90%; or at least 92%; or at least 95%; or at least 97% of the particles fall within a specified size range, for example wherein the size range is between 1 and 1,000 nm; or between 1 nm and 600 nm; or between 50 nm and 500 nm; or between 100 nm and 400 nm; or between 150 nm and 350 nm; or between 200 nm and 300 nm.

The term “microcapsule,” “microparticle” or “microsphere” as used herein refers to particles having a size (e.g., a diameter) between 1 μm and 1,000 μm; or between 1 μm and 500 μm; or between 1 μm and 100 μm; or between 1 μm and 50 μm; or between 2 μm and 30 μm; or between 3 μm and 30 μm; or between 3 μm and 10 μm. In certain embodiments, a “microcapsule composition” as used herein refers to a composition that includes particles wherein at least 30%; or at least 40%; or at least 50%; or at least 60%; or at least 65%; or at least 70%; or at least 75%; or at least 80%; or at least 85%; or at least 87%; or at least 90%; or at least 92%; or at least 95%; or at least 97% of the particles fall within a specified size range, for example wherein the size range is between 1 μm and 1,000 μm; or between 1 μm and 500 μm; or between 1 μm and 100 μm; or between 1 μm and 50 μm; or between 2 μm and 30 or between 3 μm and 30 μm; or between 3 μm and 10 μm.

Microcapsules and/or nanocapsules as described herein may be made or manufactured using any technique known in the art, including emulsification techniques (including double-emulsification techniques), spray drying techniques, water-in-oil-in-water techniques, syringe extrusion techniques, coaxial air flow methods, mechanical disturbance methods, electrostatic force methods, electrostatic bead generator methods, and/or droplet generator methods. For example, microparticles and/or nanoparticles of the present technology may be manufactured using techniques and methods similar to those described in U.S. Pat. No. 6,884,432, hereby incorporated by reference in its entirety. In certain embodiments, microcapsules or nanocapsules of the present technology may be gelatin-based; for example similar to those disclosed in Vandelli, et al., International Journal of Pharmaceutics (2001), 215:175-185. In various embodiments, microparticles and or nanoparticles include a gel or matrix having the monomers, polymers and/or polymerization initiators as described herein. The size and other properties of microcapsules and nanocapsules may be changed by altering various parameters in the production process. Freidberg et al., (2004) 282:1-18 (hereby incorporated by reference in its entirety) provides a review of procedures and compositions for microsphere manufacture, any of which procedures and compositions may be used in conjunction with microcapsules or nanocapsules of the present technology.

Compounds for Controlled Delivery

Compounds that may be controllably delivered by the methods and compositions of the present technology include any compound of which it may be desirable to control or regulate the release of For example, the compound may be a biologically active compound such as a drug, hormone, growth factor (cytokine). The compound may in certain embodiments may be a peptide or protein. In some embodiments, the compound may be a nucleic acid or based on nucleic acid. For example, in some illustrative embodiments the compound may be DNA, RNA, siRNA, an oligonucleotide a plasmid or the like. In certain embodiments the compound is one or more of: Autocrine motility factor, bone morphogenetic proteins (BMPs), epidermal growth factor (EGF), erythropoietin (EPO), fibroblast growth factor (FGF), granulocyte-colony stimulating factor (G-CSF), granulocyte-macrophage colony stimulating factor (GM-CSF), growth differentiation factor-9 (GDF9), hepatocyte growth factor (HGF), hepatoma derived growth factor (HDGF), insulin-like growth factor (IGF), migration-stimulating factor, myostatin (GDF-8), nerve growth factor (NGF) and other neurotrophins, platelet-derived growth factor (PDGF), thrombopoietin (TPO), transforming growth factor alpha (TGF-α), transforming growth factor beta (TGF-β), vascular endothelial growth factor (VEGF), placental growth factor (PIGF), Fetal Bovine Somatotrophin (FBS), IL-1, IL-2, IL-3, IL-4, IL-5, IL-6, or IL-7. In some embodiments, the compound is a hormone, for example a hormone selected from the following non-limiting list: a progestin, an estrogen, an androgen, a thyroid hormone, a growth hormone, a catacholamine hormone, and the like.

### Administration and Applications

The present technology may be used in any application that would benefit from temporally- and/or spatially-controlled delivery of compounds.

In some embodiments, the compositions of the present technology (e.g., a matrix, nanocapsule or nanocapsule such as described herein) are administered to a subject such as a mammal or a human. For example, the compound may be a biologically active compound (such as a drug, hormone or growth factor (cytokine) in which the ability to cause a sudden decrease in release and/or bioavailability is desired. In such applications, a matrix, nanocapsule or microcapsule such as described herein may be in a form suitable for administration to an animal or human.

Administration to the subject may be in any way suitable, for example, oral administration, intravenous administration, intramuscular administration, intraperitoneal administration, administration by suppositories, inhalation administration, and the like. The dosage to be administered depends to a large extent on the condition and size of the subject being treated as well as the frequency of treatment and the route of administration. As such, provided herein is a pharmaceutical product which may include a matrix, a nanocapsule, or a microcapsule as described herein may be a pharmaceutically acceptable injectable or administrateable carrier and suitable for introduction to a tissue or cells in vivo, for example in a pharmaceutically acceptable form for administration to a human and/or animal approved by an appropriate government agency. In some embodiments, a matrix, a nanocapsule, or a microcapsule as described herein may be injected subcutaneously or into a tissue of a subject. In certain illustrative embodiments where a microcapsule as described herein may be injected subcutaneously or into a tissue of a subject, the composition may be injected into the tissue such that light can penetrate the tissue such to cause crosslinking. For example, in such embodiments, the composition may be injected at depth less than 10 mm; or less than 7 mm; or less than 5 mm; or less than 2 mm; or less than 1 mm.

In some embodiments, the bioactive compound of a composition of the present technology may be a contraceptive agent (such as an estrogen) and exposing the composition to an external stimulus reduces the release of the contraceptive sufficiently for fertility to resume.

In certain embodiments, the compositions of the present technology (e.g., a matrix, nanocapsule or nanocapsule such as described herein) are administered to a cell, for example a cell in vitro cell or tissue culture conditions. In such embodiments, the compositions may be in a suitable form or buffer for in vitro cell culture procedures.

In some embodiments, the present technology is useful in tissue engineering applications. For example, microparticles or nanoparticles of the present technology may be added to a tissue scaffold and locally release a bioactive compound until crosslinking is initiated with an external stimulus. In some embodiments, the external stimulus is applied to localized area of the tissue scaffold to halt release of the bioactive compound only in the localized area. In other embodiments, the external stimulus is added to the entire scaffold to halt release at a particular time.

In certain illustrative embodiments, the technology may be applied to control release of a compound (such as a cytokine) in a bioreactor for cell and/or tissue culture and/or engineering. For example, if tissue in a certain area of a bioreactor is maturing at a different rate than in another area (e.g., due to incomplete mass transport of nutrients), cytokines can be turned off only in the matured areas, while the other regions are allowed to continue their maturation. Thus, in certain embodiments, the process may be used to assure uniform quality of grown tissue.

### Kits

The compositions (such as a matrix, as described herein), materials and components described herein may be suited for the preparation of a kit. Thus, the disclosure provides a kit useful for controlled delivery of a compound to a subject or a cell.

In one embodiment, the methods described herein may be performed by utilizing pre-packaged kits including compositions for controlled delivery (such as a matrix, a nanocapsule, a microcapsule as described herein) and/or materials to administer the controlled delivery compositions and/or materials for applying the external stimulus. The kits may contain instructions for the use of the components included in the kit; for example instructions to administer the composition to a cell or subject, and stimulate the composition with light. In some embodiments, each component of the kit can be enclosed within an individual container and all of the various containers can be within a single package.

A kit may further include a second container that includes a pharmaceutically-acceptable buffer, such as phosphate-buffered saline, Ringer's solution and/or dextrose solution. It can further include other materials desirable from a commercial and user standpoint, including other buffers, diluents, filters, stirrers, needles, syringes, and/or package inserts with indications and/or instructions for use.

The units dosage ampules or multidose containers, in which the components may be packaged prior to use, and/or may be packaged as a sterile formulation, and the hermetically sealed container is designed to preserve sterility of the formulation until use.

### EXAMPLES

The present compositions, methods and kits, thus generally described, will be understood more readily by reference to the following examples, which are provided by way of illustration and are not intended to be limiting of the present methods and kits. The following is a description of the materials and experimental procedures used in the Examples.

### Example 1

### Synthesis of VEGF-Containing Crosslinkable Microcapsules and Nanocapsules

VEGF as a bioactive compound and BDPA as a photoinitiator are mixed with PPF in the presence of organic solvet, and formed into micro- or nanocapsules using a conventional double emulsion extraction technique (see U.S. Pat. No. 6,884,432 and B. Oldham, et al, J. Biomech. Eng. (2000), 122: 289-292; hereby incorporated by reference it their entireties).

### Example 2

### Synthesis of VEGF-Containing Crosslinkable Microcapsules and Nanocapsules

VEGF as a bioactive compound and BDPA as a photoinitiator are encapsulated into PPF/microcapsules and is encapsulated into PPF/ poly(lactic-co-glycolic acid) (PLGA)-based microparticles or nanoparticles using a conventional double emulsion extraction technique (see U.S. Pat. No. 6,884,432 and B. Oldham, et al, J. Biomech. Eng. (2000), 122: 289-292; hereby incorporated by reference it their entireties).

### Example 3

### Administration and Use of VEGF-Containing Crosslinkable Microcapsules and Nanocapsules in Tissue Engineering Applications

The VEGF microcapsules and nanocapsules of Example 1 are added to a porous tissue engineering scaffold in a manner allowing the microcapsules to incorporate into the porous scaffold. The microcapsules are allowed to incubate in the scaffold and release VEGF into the growing and developing tissue to promote blood vessel growth in the tissue. Once the blood vessels in the tissue have sufficiently matured (i.e., after3-5 days), the tissue scaffold is exposed to UV light with a wavelength of about 800 nm; thus crosslinking the VEGF-microcapsules and halting VEGF release to the tissue.

### Example 4

### Administration and Use of VEGF-Containing Crosslinkable Microcapsules and Nanocapsules in Tissue Engineering Applications

Microcapsules and/or nanocapsules are made in accordance with the present technology and/or the above examples that have a contraceptive agent as the biologically active ingredient. The microcapsules and/or nanocapsules are injected into a patient at a depth of 1-10 mm under the skin. The microcapsules and/or nanocapsules release the contraceptive causing controlled infertility in the patient. Once the patient is no longer desirous of the contraceptive effects, a suitable light stimulus is applied to the patient in the area that the microcapsules and/or nanocapsules were injected causing crosslinking of the microcapsules and/or nanocapsules. Following crosslinking, the release of the contraceptive was halted such that fertility in the patient resumed.

### REFERENCES

1. Yaszemski, Michael J.; Currier, Bradford L.; Lu, Lichun; Zhu, Xun; Jabbari, Esmaiel; Kempen, Diederik, H. R., Blend, cross-linkable poly(propylene fumarate) for immobilization and controlled drug delivery, U.S. Pat. No. 6,884,432, 2005
2. Jin-Feng Xing, Xian-Zi Dong, Wei-Qiang Chen, Xuan-Ming Duan, Nobuyuki Takeyasu, Takuo Tanaka, and Satoshi Kawata, Improving spatial resolution of two-photon microfabrication by using photoinitiator with high initiating efficiency, Appl. Phys. Lett. 90, 131106 (2007)
3. TP Richardson et. al., “Polymeric system for dual growth factor delivery”, Nature Biotechnology 19 (2001) 1029-10343.
4. M.A. Vandelli, F. Rivasi, P. Guerra, F. Forni, R. Arletti, Gelatin microspheres crosslinked with D,Lglyceraldehyde as a potential drug delivery system: preparation, characterisation, in vitro and in vivo studies, International Journal of Pharmaceutics 215 (2001) 175-184.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 particles refers to groups having 1, 2, or 3 particles. Similarly, a group having 1-5 particles refers to groups having 1, 2, 3, 4, or 5 particles, and so forth. As used herein, the term “about” means in quantitative terms, plus or minus 10%.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of controlled delivery of a bioactive compound, comprising:
exposing a matrix comprising a polymer, the bioactive compound, a crosslinkable monomer and a polymerization initiator to an external stimulus;
wherein the external stimulus causes crosslinking of the matrix,
wherein the matrix is administered to a subject or a cell prior to crosslinking, and
wherein the crosslinking causes a decrease in the rate that the bioactive compound is released from the matrix.

2. (canceled)

3. The method of claim 1, wherein the rate of release of the bioactive compound from the matrix following crosslinking is less than 50% of the rate of release of the bioactive compound before crosslinking.

4.-6. (canceled)

7. The method of claim 1, wherein the matrix is exposed to the external stimulus at least 1 hour after administration.

8.-10. (canceled)

11. The method of claim 1, wherein the crosslinkable monomer is a biodegradable crosslinkable monomer.

12. The method of claim 11, wherein the crosslinkable monomer is one or more monomers selected from the group consisting of: propylene fumarate; DL-lactic-co-glycolic acid or diacrylated poly(ethylene glycol) (PEGDA) monomers.

13.-16. (canceled)

17. The method of claim 1, wherein the polymerization initiator is a photoinitiator.

18. (canceled)

19. The method of claim 1, wherein the polymerization initiator is one or more polymerization initiators selected from the group consisting of 2-hydroxy-1-[4-(2-hydroxyethoxy)phenyl]-2-methyl-1-propanone (12959); 9,10-bis-pentyloxy-2,7-bis[2-(4-dimethylamino-phenyl)-vinyl]anthracene (BPDPA) or a mixture of riboflavin and L-arginine.

20. The method of claim 1, wherein the external stimulus is light.

21. (canceled)

22. The method of claim 20, wherein the light is applied to a localized area in the subject.

23. (canceled)

24. The method of claim 1, wherein the bioactive compound is a cytokine.

25. The method of claim 1, wherein the bioactive compound is VEGF.

26. The method of claim 1, wherein the matrix is in microcapsules or nanocapsules.

27. (canceled)

28. The method of claim 1, wherein the matrix is administered to a subject in vivo.

29. (canceled)

30. A composition comprising microcapsules or nanocapsules for controlled delivery of a bioactive compound wherein the composition comprises:
a matrix comprising a polymer configured to release the bioactive compound,
one or more crosslinkable monomers, and
a photoinitiator polymerization initiator configured to initiate polymerization of the crosslinkable monomer in response to an external stimulus.

31. The composition of claim 30, wherein the crosslinkable monomer polymerizes in response to stimulation with light.

32. The composition of claim 30, wherein the crosslinkable monomer is a biodegradable crosslinkable monomer.

33. The composition of claim 32, wherein the crosslinkable monomer is one or more monomers selected from the group consisting of propylene fumarate; DL-lactic-co-glycolic acid; or DL-lactic-co-glycolic acid or diacrylated poly(ethylene glycol) (PEGDA) monomers.

34.-36. (canceled)

37. The composition of claim 30, wherein the polymerization initiator is a two-photon photoinitiator.

38. The composition of claim 30, wherein the polymerization initiator is one or more polymerization initiators selected from the group consisting of 1,2-hydroxy-1-[4-(2-hydroxyethoxy)phenyl]-2-methyl-1-propanone (I2959); 9,10-bis-pentyloxy-2,7-bis[2-(4-dimethylamino-phenyl)-vinyl]anthracene (BPDPA) or a mixture of riboflavin and L-arginine.

39. The composition of claim 30, wherein the bioactive compound is a cytokine.

40. The composition of claim 30, wherein the bioactive compound is a growth factor.

41. The composition of claim 30, wherein the bioactive compound is VEGF.

42.-47. (canceled)
